(19) United States
(12) Patent Application Publication
(10) Pub. No.: US 2005/0084923 A1
(43) Pub. Date: Apr. 21, 2005

(54) Methods for cultivating and analyzing microbial individual cell cultures

(76) Inventors: Peter-Juergen Mueller, Jena (DE); Martin Roth, Jena (DE); Monika Hilliger, Jena (DE); Stephan Hilliger, Wurzburg (DE); Ingrid Groth, Jena (DE); Christel Kummer, Cospeda (DE); Karin Martin, Grosslobichau (DE); Volker Schoeckh, Rothenstein (DE); Jose Metze, Heiligenstadt (DE); Michael Kohler, Golmsdorf (DE); Thomas Henkel, Jena (DE); Gunter Gastrock, Heiligenstadt (DE); Karen Lemke, Gottingen (DE)

Correspondence Address: JORDAN AND HAMBURG LLP, 122 EAST 42ND STREET, SUITE 4000, NEW YORK, NY, 10168, US

(21) Appl. No.: 10/489,141

(22) PCT Filed: Sep. 13, 2002
(86) PCT No.: PCT/DE02/03451

(30) Foreign Application Priority Data
Sep. 14, 2001 (DE) 101 45 568.2

(51) Int. Cl. C12Q 1/04; C12N 1/20

(52) U.S. Cl. 435/34; 435/252.1

(57) ABSTRACT

The invention relates to methods for individually separating microorganisms from a suspension or culture, parallel cultivation of individual cells and analyzing the metabolic performances thereof. The invention enables basic microbial operations such as media optimization, screening according to images of novel natural substances and special metabolic performances, qualitative and quantitative detection of the effects of nutrient substrates, effectors and active substances including the media optimization and selection of microorganism clones with specific properties from large populations according to mutagenesis, transformation, transfection and genetic processing in addition to the detection of microbial contaminations to be carried out. One advantage of the invention is that it can be applied when microorganisms having outstanding properties can be respectively obtained as individual cells or individual organisms from a large population and can be characterized as pure cultures or when the effect of influencing variables can be examined in many fully comparable cultures.

One of the most frequently performed basic operations in microbiology, the separation and parallel cultivation of individual organisms, can be performed with the invention with a previously unachieved number of individual microbes.

The goal of the separation is for instance to find microorganisms with outstanding properties. Microorganisms with outstanding properties frequently occur in microorganism cultures and populations in very small numbers, measured against the total number. They occur consistently in every microbial population by spontaneous mutation, they are produced deliberately and artificially by mutagenesis, transfection, transformation, and genetic engineering methods or enter the culture by way of contamination.

The search (screening) for new microorganisms with novel, better properties, but also the evidence of microorganisms with pathogenic or harmful properties, occurs in samples that are obtained as suspensions of natural or anthropomorphously influenced locations or products, for instance soils or foods, and in aqueous habitats, for instance waste water facilities, or from living higher organisms.

The invention opens a path for finding individual microbial organisms or individual microbes with novel and/or special abilities or properties in a large—with respect to the microorganisms—homogeneous or heterogeneous population and thus for being better able to utilize the great potential of the microbial abilities.

Likewise, the invention can be advantageously employed when the viability of microorganisms is employed as an indicator for qualitative or quantitative determination of nutrient substrates or effectors of growth and metabolism, e.g., of antibiotics or essential nutrient components. It is also possible to optimize nutrient compositions of the media used for cultivation.

The possibility arises to perform these examinations with their very high number of monoclonal cells or cultures in the form of micro-pure cultures.

The invention can be used wherever microbial abilities or effects are sought, applied, improved, or analyzed, for instance in biotechnology, genetic engineering, medical microbiology, pharmaceuticals, microbiology, and foods/environmental microbiology.

The production of pure cultures and/or monoclonal cultures and/or non-contaminated cultures is a basic operation in microbiology [1]. A pure culture comprises the progeny of a single cell. Their cells have the same growth and metabolic properties. For obtaining pure cultures, it is necessary to isolate individual cells for inoculating the cultures.

However, finding and obtaining interesting individual microorganisms from a submerged culture (liquid culture) constituting a very large number of microorganisms or from a cell suspension represents a technical scientific problem that has not been satisfactorily resolved in the past [2]. Previously this task was only performed with inadequate methods or by plating highly diluted culture suspensions on agar surfaces, or mechanical manipulation was used to isolate individual cells, for instance using a so-called laser pipette [3]. Applying mechanical cell sorting methods in the current state of the art is still very time-consuming and not practical because of the great number of organisms to be separated.

Theoretically, very small cultivation vessels could be employed for performing large cultivations in parallel. The principle of using microtechnically produced cavities and channels for biotechnological cultivations has already been suggested [4]. Initial trials for practical employment of microsystem technology demonstrated that parallel inoculation and cultivation of 16 microcultures (Escherichia coli) is possible on a 1-μL scale [5]. Pipetting of microorganisms can be realized with portioners [6].

Separation of the microorganisms to be used for inoculation from a large population of cells, e.g., 10⁵ to 10⁸, has not been performed in the past due to the substantially greater number of microculture vessels required then.

Microorganisms are enclosed in gel microdroplets (GMD) for a variety of biotechnical applications. A special system of nozzles divides a suspension containing microorganisms and a water-soluble, gel-forming material into the smallest possible drops, which contain individual cells, and these are then consolidated into GMDs or are microcapsulated [7-9]. The GMDs are incubated in a liquid nutrient medium for cultivating the microorganisms. The growth and selected properties of the microorganisms in the individual GMDs can be detected using various methods [7-9]. This method is disadvantageous in that rapidly growing microorganisms can exit the GMDs into the surrounding nutrient solution after just a brief period of cultivation and thus contaminate all of the other GMDs. Therefore this method is not suitable, especially for cultivating, characterizing, and isolating various rapidly growing microorganisms or cells. A further disadvantage is that multiple measurements of the individual GMDs and the associated data acquisition are very difficult to realize technically.

Classic methods are used nearly exclusively in the isolation of mutants, selectants, contaminants, or genetically engineered microorganisms. The cell populations are diluted such that after applying the diluted bacterial suspensions to the surface of agar cultures, separate colonies or individual colonies occur that each derive from a single microorganism cell.

In addition, frequently selective conditions are produced by the deliberate choice of substrate or the addition of growth effectors with which only the desired microorganisms can grow.

In genetic engineering, those genes that are to be carried forward are coupled to marker genes. The marker genes are frequently genes that resist antibiotics. This means that only those clones that contain the gene that was carried forward grow in cultures to which antibiotics were added.

In many cases there is no simple opportunity to recognize and obtain, that is, to isolate, in a simple and direct manner, the interesting microorganisms that are generally present in smaller numbers.

As an example, the state of the art shall be illustrated using the procedure in a primary screening, the search for new microorganisms with new abilities [10]. Extrapolations of counts in extracts from soil samples demonstrated that a maximum of only approximately 1 to 10% of the microorganisms, of identical and different types, occurring with an average number of a total of approximately 10⁶ to 10⁸ per gram of soil sample, are found using these traditional screening methods.

In traditional primary screening, the samples are suspended in a buffer or water in order to obtain defined microbial suspensions (submerged samples). The concomitant solids, for instance soil, are separated and the liquid supernatant (extract) containing the microorganisms that have been rinsed off is diluted (dilution steps) until after subsequent application on agar surfaces emerging growing individual microbial colonies occur that are isolated or separated from one another by growth-free zones. These are isolated and checked for interesting abilities and properties. The primary goal of the dilution is to obtain separate and uncontaminated colonies (pure cultures). In a typical primary screening procedure, 1 g of a soil sample (calculated as dry weight) is suspended with 10 mL of a buffer, saline solution, or water, and diluted using dilution steps approximately 10⁶-fold with well-colonized garden soils. Petri dishes with agar media are each inoculated with 0.1 mL extract dilution at the dilution stage at which individual colonies occur. What this procedure leads to is that only those microorganisms that are still present after the dilution in 0.1 mL extract dilution can grow on the agar surface.

After incubation, the presence of for instance 10⁶ to 10⁸ colony-forming units per g of soil dry mass can be calculated based on the grown colony counts and the dilution steps used. However, cultivation of this large number of microorganisms is not possible with the method used. From the great number of microorganism colonies grown, subsequently generally transferring and further cultivation is performed, taking into account morphological properties of selected colonies. The selected clones are then examined for new metabolic performances in a secondary screening. Microorganism species that are present for instance in a 100-times lower concentration in the soil sample are found with a 100-times lower probability with the described procedure.

In addition to the loss of microorganisms in the sample material that is caused by the dilution regime, there are additional factors opposing comprehensive results. These factors are found in the physiology of the microorganisms.

Approximately 90% of the microorganisms present in the soil sample are calculated in a lump sum as “non-cultivatable” microorganisms. Non-cultivatable means that these microorganisms do not grow under the selected growth conditions. In order to cultivate them, the growth conditions must be adapted to the particular requirements of the microorganisms in terms of nutrient media and physical parameters. There is the problem that a portion of the microorganisms in their biotope/ecosystem are in a physiologically inactive condition (dormancy) (K strategies). They are viable, but are not cultivatable under the conventionally employed standard conditions or during the cultivation periods used. Other microorganisms (r strategies) grow very rapidly. One reason for the failure to find a majority of the microorganisms could be that the K strategies or the “non-cultivatable” microorganisms frequently do not grow among the microorganisms or are overgrown by r strategies. There have also been indications that the growth of microorganisms is regulated by growth factors. It is a known phenomenon that now and then cultures that are incubated too thinly do not grow. Growth is induced if a small quantity of the filtrate from a growing culture of a microorganism is added to the inoculated culture.

It is the goal of the invention to perform the basic microbiological operations of cell separation and single-cell cultivation in parallel in larger numbers in a simple manner. The microbial specification shall be taken into account that pure cultures would actually be required for many applications, but because of the high individual counts in the cultures have not been easily realizable in the past. What this led to in the past for instance was that, in a microorganism population in very limited numbers, types present with interesting or excellent properties are not found. Submerged microbial cultures shall therefore be treated such that to the extent possible each of the microorganisms present in a cell suspension shall obtain the opportunity to grow as an individual organism in a separate cultivation sphere as a pure culture or microculture. In addition, the situation shall be prevented in which in screening using the traditionally necessarily employed dilution steps the practical isolatable individual count is reduced in great measure.

In order to detect, find, and isolate microorganisms with unusual properties or microbial infections or novel or rare microorganisms, including the K strategies and the non-cultivatable microorganisms/microorganisms that are difficult to cultivate, or in order to examine the effect of effectors using a great number of parallel growth trials in a manner that can be statistically evaluated, the inventive object presents itself of developing methods with which all microorganisms in an aqueous microorganism suspension that contains a great number of identical or different microorganisms can be cultivated in the form of pure cultures.

This object includes the development of a method for separating all microorganisms present in a culture by using the options available through microsystem engineering. In addition, where appropriate, growth conditions should be able to be varied such that growth is promoted for separated microorganisms with selected properties, but other undesired microorganisms cannot grow or can only grow to a limited extent.

For achieving these objects, in accordance with the invention a method for parallel cultivation of microorganisms is suggested that is characterized in that nutrient substrates and/or effectors and/or microbial metabolites are added to a homogeneous or heterogeneous microorganism population constituting a suspension or culture relieved of coarse solids, then a volume v of the microbial suspension, which contains N microorganisms, is divided with a portioner into n₁ partial volumes, whereby the number n₁ is selected between N and 100N, preferably between N and 10N, then the partial volumes, where appropriate with the addition of nutrient substrates and/or effectors for inoculation of n₂ separate microcultures, are used in microareas or microcavities, whereby n₂ is greater than or equal to n₁, then the microcultures are incubated and during the growth where appropriate additional nutrient substrates and/or effectors and/or metabolites are added and physiological parameters and the growth of the individual microcultures is detected with appropriate measuring methods.

Microorganisms in the sense of this invention are prokaryotic and eukaryotic cells, whereby the cells can be present individually and/or as cell clusters/cell aggregates and/or as tissue fragments. Among prokaryotic cells are bacteria and blue algae; the eukaryotic cells include yeasts, fungi, animal cells, and plant cells.

The number N/v is for instance determined microscopically by counting in a bacteria or blood count chamber or by other methods known per se. The number n, in accordance with the invention is between 10⁴ and 10⁸.

The volume of the partial volumes arising during the separation is between 0.1 nL and 1 μL, the microcavities and microcultures receiving them have a volume of 0.1 nL to 10 μL.

All of the elements essential for the structure of the microorganism cells (C, 0, H, N, S, P, K, Na, Ca, Mg, Fe) and so-called trace elements are added as nutrient substrates in a form that is available for the cells.

In addition, in accordance with the invention effectors of microbial growth are added to microcultures, such as for instance growth activators or growth inhibitors, enzyme inhibitors or enzyme activators, antibiotics, cytokine, enzymes, vitamins, amino acids, antimetabolites, and microbial metabolites.

Intentionally adding effectors of microbial growth can suppress the growth of undesired microorganisms or can promote the growth of desired microorganisms, or can induce certain product formations or metabolic abilities of the microcultures.

Effectors of microbial growth influence growth positively or negatively. The addition of antibiotic substances corresponding to the type of pure culture and depending on its concentration leads to inhibition of growth of non-resistant microorganisms. For instance, the addition of antifungal antibiotics prevents the growth of fungi that have the property of overgrowing bacterial microcultures, which is very disadvantageous for the inventive process. When bacteria are to be cultivated, therefore, antifungal-acting substances are added in order to prevent fungi that disturb growth.

The addition of antimetabolites inhibits growth using a negative influence on metabolic paths. In addition, in another type of cultivation, a high concentration of one or more antibiotics can be added that only act on growing microorganisms and inhibit them (e.g., a penicillin derivative). Then the culture is centrifuged and the antibiotics are removed with the supernatant.

Growth-promoting metabolites are added in pure form or in culture filtrates of prokaryotic and/or eukaryotic cell cultures and/or in concentrates thereof and/or in extracts of prokaryotic and/or eukaryotic cell cultures. This stimulates the growth of microorganisms that are difficult to cultivate, for instance.

The technical realization of the microcultures occurs inventively in microcavities. Inventively adequate methods are removal of solids, separation, portioning, inoculation, nutrient supply including oxygen supply and addition of microbial metabolites and effectors of microbial growth, production of selective growth conditions, and measurements of growth and product formation. The separation procedure is preferably closely connected technically with the microcultivation procedure. The conditions for microbial cultivation, known per se, such as maintaining constant physiologically tolerated temperatures and acidity, are included in the methods known per se. Sterility of the apparatus is achieved in a manner known per se by heating with steam to 121° C., by dry heating to temperatures greater than 150° C., by chemical sterilization, or by sterilization by means of radiation.

The details of the inventive procedure are explained in the following.

A simple buffer or water is added to a soil sample, for instance, and after vigorous shaking using a centrifuge the solids are sedimented, removed, and then the microorganisms are obtained as a pellet using the centrifuge. The pellet is suspended in a medium that contains all essential nutrient substrates and where necessary effectors of microbial growth.

Undesired, rapidly growing bacteria are killed in that one or a plurality of antibiotics are added that act only on growing microorganisms (e.g. penicillin). After for instance 4 hours of incubation, the culture is centrifuged and the antibiotics are removed in the supernatant.

Microcultivations occur inventively in microcavities that are completely or partially filled with the partial volumes obtained by separation. By using the opportunities offered by microsystem engineering, the inventive procedure provides an advantageous novel path to system-appropriate treatment of the individual microbes, which in this context are generally particularly high in number.

The inventively employed microcavities are generally arranged in two dimensions. The volume of the microcavities is between 0.1 nL and 10 μL.

The separation of the microorganisms present in the suspension or separation of microorganisms is realized by filling microcavities in the form of microcapillaries or microcapillaries arranged in an array with a volume equivalent between 0.1 nL and 1 μL.

Cultivation of the separated microorganisms occurs in this method in microcapillaries in microcultures that are separated from one another and that have a volume between 0.1 nL and 1 μL.

For separating the microorganisms, in particular a miniaturized thermally controlled liquid switch or a miniaturized liquid switch in combination with a microinjection unit or a pneumatically driven liquid switch is used.

Alternatively, a switch based on electrical principles is employed that is embodied as an electrostatic or electromagnetic or dielectrophoretic switch.

By periodically changing the volume flow rate with gas bubbles or with separating liquids that are not miscible with water, liquid segments are obtained for which there is a probability of <5% that they contain more than one cell per segment. A single capillary is filled with a plurality of such liquid segments and contains the described number of separated individual compartments, each with one cell.

Pulsing fluctuations in pressure in the capillaries improves the mixing or oxygen transition via the open end of the capillaries.

The microcultivation can inventively also occur in a plurality of capillaries, whereby each capillary represents a microcavity. Filling with culture liquid, i.e., the inoculation process, occurs by feeding or passively by suctioning using capillary forces. A pulsing change in pressure at one end of the capillary produces and back and forth movement by the culture liquid in the capillary and thus improves mixing or oxygen transition via the open end of the capillary.

A one-dimensional microculture variant is employed by inventive use of a liquid system with serial sample separation. The technical arrangements and systems known from flow injection analysis are used for microbial cultivation. Parallel multiple arrangements increase the number of microcultures.

Microcapillaries introduced into chips act as storage and culture spaces. A capillary length of approximately 1 m is situated in one single chip of something more than 2 cm². The microcultures are separated from one another in the capillaries by a barrier liquid.

Approximately 5,000 samples with individual volumes of approximately 0.1 nL are cultivated in the capillary that is 1 m in length. 200 of these chips are used to receive approximately 1 million microcultures. The total volume of these chips is less than 100 mL. Given a significant increase in the individual volumes by a factor of 10 (to approximately 1 nL), the total volume of all of the chips is one Liter.

Loops of inexpensive tube material for storing the samples that are not segmented by liquid sections are employed for one-dimensional cultivation of samples whose total volume is greater than one Liter.

What is advantageous in the inventive separation of the microorganisms contained in suspensions or cultures into volume equivalents is that the division of each volume equivalent contains on average one individual microorganism. Each of the separated microorganisms can grow very rapidly or can start growing only after an extended delay phase, corresponding to its growth behavior, without the slowly growing individual microorganisms being overgrown by more rapidly growing individual microorganisms.

In a certain limited number of cases, in particular when n, =N, they contain one microorganism, more than one microorganism, or no microorganisms. The blank equivalents can be detected based on lack of growth.

For system control, at the time of microorganism separation and their introduction into a microcavity or a microarea, the coordinate allocation is stored and registered on a fixed storage medium, whereby unambiguous allocation is possible at any time.

For separating the microorganisms, a system of portioners is used in which the volume of the individually dispensed drops is between 0.1 nL and 1 μL and 1 drop is dispensed into each microarea or microcavity.

The drops are dispensed by means of volume pulse optimizing without formation of splashes.

In another embodiment, a portioner is used to separate the microorganisms, which is provided with a particle or cell counting device and which dispenses the liquid containing the microorganisms in individual drops of 0.1 nL to 1 μL volume and stops filling a receiving position either when its maximum fill volume has been achieved or when a drop containing a cell has been placed.

Alternatively, a piezoelectrically controlled portioner can be employed to separate the microorganisms, whereby the drop frequency and the drop size are adapted to the feed movement of the positioning device and to the cell concentration, interior volume, and spatial frequency of the sample receiving regions such that there is a probability of <5% that more than one cell is dispensed per receiving position.

In one further variant for separating the microorganisms, a pneumatically or electropneumatically controlled portioner is employed, whereby the drop frequency and the drop size are adapted to the feed movement of the positioning device, and to the cell concentration, interior volume, and spatial frequency of the sample receiving regions such that there is a probability of <5% that more than one cell is being dispensed per receiving position.

In one further inventive embodiment, nanotiter plates [11] with cavities in the volume range of 0.01 to 500 nL per cavity are employed for compartmented cultivation of microorganisms. After separation, the microcultures are cultivated in microcavities that are arranged in an array at a distance from one another that is equal to or less than 1.8 mm.

Suitable for this are in particular nanotiter plates with microcavities that have a conical or a cylindrical or a spherical segment or a prismatic, pyramid, double or multiple pyramid shape.

After separation, the microcultures are cultivated in the chambers of nanotiter plates.

Gas and nutrient supply of the microcultures can occur using a micropore membrane, the pore width of which is preferably between 0.1 μm and 4 μm and the membrane thickness of which is between 0.2 μm and 10 μm, so that the cells are retained.

For this purpose, the nutrient supply in the microcultures can occur using a micropore membrane or a nanopore membrane that is covered on the supply side by a microliquid channel system.

In certain embodiments the microcavities of the nanotiter plates obtain common supply via the micropore or nanopore membranes, while effectors of microbial growth are optionally applied to the microcavities from above.

Likewise, the supply of the microcultures can occur via a micropore membrane with one or a plurality of microchannels that are incorporated into a (micro)flow injection arrangement such that the effect of effectors or nutrient substrates can be tested simply and serially by injection into the perfusion channel.

The production of the microliquid channels providing the supply, which carry a micropore membrane, is realized using a series of one isotropic and one anisotropic etching step in silicon.

For improving manageability and avoiding liquid disturbances during filling, the stays between the microchambers can be provided with a water-repellant surface coating.

The prerequisite for the selection of microorganisms with certain properties is the analytic access to physiological and culture parameters. In accordance with the invention, the complete or partial use of the methods and design features cited in the following is provided.

Electroimpedance spectroscopy (EIS) is preferably employed for analyzing physiological parameters and for measuring the growth in each of the microcultures.

The kinetics of the culture parameters pH, pO₂, pCO₂, are detected by means of spectroscopic methods prior to and after the flowing of the diffusive supply of the microorganisms present in the suspension.

Alternatively, the growth of the microcultures is tracked microturbidometrically or photometrically.

Chip chambers with at least 2 transparent side walls parallel to one another and arranged plane-parallel are used for measuring the growth of the microcultures.

These plane-parallel side walls are optionally partially equipped with a highly reflecting thin film, whereby microstructured windows are inserted therein for coupling and decoupling the light.

The growth of the microcultures is tracked using the increase in the flow resistance during movement of the small liquid volumes based on the increasing total viscosity of the liquid containing the cells.

Alternatively the growth of the microcultures is tracked using the amplification of the deflection, focusing, or defocusing of a non-absorbed laser beam during heating of the liquid containing the cells using a laser beam partially absorbed by the cells.

For detecting the radiation position of the measuring light, receiver double cells are used and with their assistance the differences in the asymmetries of the light intensities corresponding to the individual positions in the local culture regions are used as measurement variables.

Exemplary Embodiment

A nutrient medium with 2 g yeast extract, 20 g malt extract, and 10 g glucose per liter is inoculated with Saccharomyces cerevisiae yeast cells. After 18 h incubation at 30° C. as a standing culture, the number of the yeast cells located in the culture is determined using a microscopic counting chamber by counting using a microscope. Then the suspension is diluted and plated on an agar medium (2 g yeast extract, 20 g malt extract, and 15 g agar per liter, pH 6.2) in 10-cm Petri dishes such that approx. 25 cells are applied per cm². The Petri dishes are incubated 3 hours at 30° C.

For compartmented cloning, cavities of nanotiter plates are filled with liquid agar medium (2 g yeast extract, 20 g malt extract, 6 g agar, pH 6.2) and covered with positively fitted silicon stamps. Once the agar has hardened, the silicon stamps are removed and replaced with a second silicon stamp, to which cells from the precultivated agar plates were previously transferred by stamping. Prior to stamping, the pre-cultivated agar plates are dried 20 minutes at 37° C. and the temperature of the silicon stamp is brought to 37° C. The inoculated silicon stamp is pressed onto the nanotiter plate by means of a clamping apparatus such that the stays of the nanotiter plate are sealed by the silicon stamp. The nanotiter plates thus inoculated are incubated at 30° C. The growth in the cavities of the nanotiter plates is tracked by mean of turbidity measurement using a reflected light microscope. The removal of clones for further cultivation and testing occurs by means of a sterile inoculation needle, destroying the membrane situated on the bottom of the nanotiter plate.

Nanotiter plates made of silicon with a metal-reinforced bottom membrane are used for the cultivation. The chamber opening is 800×800 μm in a 1-mm grid. The bottom width is approx. 150×150 μm, the total chamber volume is approximately 150 nL. (Manufacturer: Institute of Physical High Technology e.V., Jena, Biotechnical Microsystems Department, Winzerlaer Strasse 10, 07745 Jena, http://www.ipht-jena.de). Silicon stamps are produced by molding nanotiter plates with identical geometry and to 100 μm reduced etching depth. Commercially available additive crosslinking silicon is used as molding material (manufacturer, e.g., Sylgard).

### Literature Citations

[1] Chan, E. C. S., Pelczar, M. J., Krieg, N. R. (1993) Use of pure cultures—basic requirement for the study of microorganisms, in: Laboratory Exercise in Microbiology, Chan, E. C. S., Pelczar, M. J., Krieg, N (eds.). McCraw-Hill, New York, 123-152
[2] Gottschall, J. C., Harder, W., Prins, R. A. (1992), In: The Procaryotes. Balows A., Trüper, H. G., Dworkin, M., Harder, W., Schleifer K.-H. (eds.). Springer, New York 149-196
[3] Huber, R. (1999) The laser pipette as the basis for individual cultivations. Biospektrum 4, 289-291
[4] Kroy, W., Seidel, H., Dette, E., Deimel, P., Binder, F., Hilpert, R. Koniger, M. (1990) DE 3915920
[5] Schober, A., Schlingloff, G., Thamm, A., Vetter, D., Thomandl, D., Gebinoga, M., Kiel, H. J., Scheffler, C., Dohring, M., Köhler, J. M., Mayer, G. (1996) System integration of microsystems/chip elements in miniaturized automats for high-throughput synthesis and screening in biology, biochemistry, and chemistry. Micro System Technologies (Potsdam October 1996), Proc. p. 705, presentation
[6] Schober, A., Günther, R., Schwienhorst, A., Doring, M., Lindemann, B. F. (1993) BioTechniques 15, 324
[7] Weaver, J. C. (1981) U.S. Pat. No. 4,401,755
[8] Williams, G. B., Weaver, J. C., Demain, A. L. (1990) Rapid microbial detection and enumeration using gel microdroplets and colorimetric or fluorescence indicator systems. J. Clin. Microbiol. 28, 1002-1008
[9] Manome, A., Zhang, H., Tani, Y., Katsuragi, T., Kurane, R., Tsuchida, T. (2001) Application of gel microdroplet and flow cytometry techniques to selective enrichment of non-growing cells. FEMS Microbiol. Lett. 197, 29-33
[10] Omura, S. (1986) Philosophy of new drug discovery, Microbiol. Reviews 50, 259-279
[11] Mayer, G., Wohlfart, K., Schober, A., Köhler, J. M. (1999) Nanotiterplates for synthesis and screening, in: Microsystem technology: a powerful tool for biomolecular studies.

Köhler, J. M., Mejevaia, T., Saluz, H. P. (eds.). p. 75-128, Birkhauser Basel

1. Method for parallel cultivation of microorganisms characterized in that nutrient substrates and/or effectors and/or microbial metabolites are added to a homogeneous or heterogeneous microorganism population constituting a suspension or culture relieved of coarse solids, then a volume v of said microbial suspension, which contains N microorganisms, is divided with a portioner into n₁ partial volumes, whereby the number n₁ is selected between N and 100N, preferably between N and 10N, then said partial volumes, where appropriate with the addition of nutrient substrates and/or effectors for inoculation of n₂ separate microcultures, are used in microareas or microcavities, whereby n₂ is greater than or equal to n₁, then said microcultures are incubated and during the growth where appropriate additional nutrient substrates and/or effectors and/or metabolites are added and physiological parameters and the growth of said individual microcultures is detected with appropriate measuring methods.

2. Method in accordance with claim 1, characterized in that prokaryotic and eukaryotic cells are defined as microorgansims.

3. Method in accordance with claim 1 or 2, characterized in that equivalent partial volumes are produced and the number n₁ is between 10⁴ and 10⁸.

4. Method in accordance with claim 1 or 2, characterized in that the volume of said partial volumes arising during said separation is between 0.1 nL and 1 μL and the volume of said microcavities receiving them and the volume of said resulting microcultures receiving them is between 1 nL and 10 μL.

5. Method in accordance with claim 1 or 2, characterized in that culture filtrates, containing growth-promoting metabolites, of prokaryotic and/or eukaryotic cell cultures and/or concentrates thereof and/or extracts of prokaryotic and/or eukaryotic cell cultures are added to said microcultures.

6. Method in accordance with claim 1 or 2, characterized in that effectors, such as growth activators or growth inhibitors, enzyme inhibitors or enzyme activators, active substances, antibiotics, cytokines, vitamins, amino acids, enzymes, or antimetabolites, are added to said microcultures.

7. Method in accordance with claim 1 or 2, characterized in that said separation of said microorganisms present in said suspension or separation of said microorganisms occurs by filling microcavities in the form of microcapillaries or microcapillaries arranged in an array with partial volumes of said resulting microcultures between 0.1 nL and 1 μL.

8. Method in accordance with claim 7, characterized in that cultivation of said separated microorganisms in microcapillaries occurs in microcultures that are separated from one another and that have a volume between 0.1 nL and 1 μL.

9. Method in accordance with claim 7, characterized in that a miniaturized thermally controlled liquid switch is used for separating said microorganisms.

10. Method in accordance with claim 7, characterized in that for separating said microorganisms a miniaturized liquid switch is used in conjunction with a microinjection unit or a pneumatically driven liquid switch.

11. Method in accordance with claim 7, characterized in that for separating microorganisms a switch based on electrical principles is used that is embodied as an electrostatic or electromagnetic or dielectrophoretic switch.

12. Method in accordance with claim 7, characterized in that in a closed microcapillary system, periodically changing the volume flow rate with gas bubbles or with separating liquids that are not miscible with water produces liquid segments for which there is a probability of <5% that they contain more than one cell per segment.

13. Method in accordance with claim 7, characterized in that the mixing or oxygen transition is improved via the open end of said capillaries by pulsing fluctuations in pressure in said capillaries.

14. Method in accordance with claim 1 or 2, characterized in that after separation said microcultures are cultivated in microcavities that are arranged in an array at a distance from one another that is equal to or less than 1.8 mm.

15. Method in accordance with claim 14, characterized in that said microcavities have a conical or a cylindrical or a spherical segment or a prismatic, pyramid, double or multiple pyramid shape.

16. Method in accordance with claim 14, characterized in that said microcultures are cultivated in the chambers of nanotiter plates.

17. Method in accordance with claim 14, characterized in that the nutrient supply of said microcultures can occur using a micropore membrane, the pore width of which is preferably between 0.1 μm and 4 μm and the membrane thickness of which is between 0.2 μm and 10 μm, so that said cells are retained.

18. Method in accordance with claim 14, characterized in that the nutrient supply in said microcultures occurs using a micropore membrane or a nanopore membrane that is covered on the supply side by a microliquid channel system

19. Method in accordance with claim 14, characterized in that all microcavities of said nanotiter plates obtain common supply via said micropore or nanopore membranes and effectors of growth are applied to said microcavities from above.

20. Method in accordance with claim 14, characterized in that the supply of said microcultures occurs via a micropore membrane using one or a plurality of microchannels that are incorporated into a (micro)flow injection arrangement such that the effect of effectors or nutrient substrates can be tested simply and serially by injection into the perfusion channel.

21. Method in accordance with claim 14, characterized in that said microliquid channels providing the supply carry a micropore membrane that is produced using a series of one isotropic and one anisotropic etching step in silicon.

22. Method in accordance with claim 14, characterized in that the stays between said microchambers are be provided with a water-repellant surface coating.

23. Method in accordance with claim 1 or 2, characterized in that the electroimpedance spectroscopy (EIS) method is employed for analyzing physiological parameters and for measuring the growth in each of said microcultures.

24. Method in accordance with claim 1 or 2, characterized in that the kinetics of the culture parameters pH, pO₂, pCO₂, are detected by means of spectroscopic methods prior to and after the flowing of the diffusive supply of said microorganisms present in said suspension.

25. Method in accordance with claim 1 or 2, characterized in that the growth of said microcultures is tracked microturbidometrically or photometrically.

26. Method in accordance with claim 1 or 2, characterized in that chip chambers with at least 2 transparent side walls parallel to one another and arranged plane parallel are used for measuring the growth of said microcultures.

27. Method in accordance with claim 26, characterized in that said side walls that are plane-parallel to one another are partially equipped with a highly reflecting thin film, whereby microstructured windows are inserted therein for coupling and decoupling the light.

28. Method in accordance with claim 1 or 2, characterized in that the growth of said microcultures is tracked using the increase in the flow resistance during movement of the small liquid volumes based on the increasing total viscosity of said liquid containing said cells.

29. Method in accordance with claim 1 or 2, characterized in that the growth of said microcultures is tracked using the amplification of the deflection, focusing, or defocusing of a non-absorbed laser beam during heating of said liquid containing said cells using a laser beam partially absorbed by said cells.

30. Method in accordance with claim 1 or 2, characterized in that for detecting the radiation position of the measuring light, receiver double cells are used and with their assistance the differences in the asymmetries of the light intensities corresponding to the individual positions in the local culture regions are used as measurement variables.

31. Method in accordance with claim 1 or 2, characterized in that for system control, at the time of microorganism separation and their introduction into a microcavity or a microarea, the coordinate allocation is stored and registered on a fixed storage medium, whereby unambiguous allocation is possible at any time.

32. Method in accordance with claim 1 or 2, characterized in that for separating said microorganisms a system of portioners is used in which the volume of the individually dispensed drops is between 0.1 nL and 1 μL and 1 drop is dispensed into each microarea or microcavity.

33. Method in accordance with claim 1 or 2, characterized in that said drops are dispensed by means of volume pulse optimizing without formation of splashes.

34. Method in accordance with claim 1 or 2, characterized in that used to separate said microorganisms is a portioner that is provided with a particle or cell counting device and that dispenses said liquid containing said microorganisms in individual drops of 0.1 nL to 1 μL volume and stops filling a receiving position either when its maximum fill volume has been achieved or when a drop containing a cell has been placed.

35. Method in accordance with claim 1 or 2, characterized in that a piezoelectrically controlled portioner is employed to separate said microorganisms, whereby the drop frequency and the drop size are adapted to the feed movement of said positioning device and to the cell concentration, interior volume, and spatial frequency of the sample receiving regions such that there is a probability of <5% that more than one cell is dispensed per receiving position.

36. Method in accordance with claim 1 or 2, characterized in that a pneumatically or electropneumatically controlled portioner is employed for separating said microorganisms, whereby the drop frequency and the drop size are adapted to the feed movement of said positioning device, and to the cell concentration, interior volume, and spatial frequency of the sample receiving regions such that there is a probability of <5% that more than one cell is being dispensed per receiving position.
